(19) United States
(12) Patent Application Publication
(10) Pub. No.: US 2004/0102692 A1
(43) Pub. Date: May 27, 2004

(54) Magnetic resonance imaging system and methods for the detection of brain iron deposits

(75) Inventors: John Frederick Schenck, Voorheesville, NY (US); Abdalmajeid Musa Alyassin, Niskayuna, NY (US); Harvey Ellis Cline, Schenectady, NY (US); William Edward Lorensen, Ballston Lake, NY (US); David Charles Alsop, Newton, MA (US)

Correspondence Address: GENERAL ELECTRIC COMPANY, GLOBAL RESEARCH, PATENT DOCKET RM. BLDG. K1-4A59, SCHENECTADY, NY, 12301-0008, US

(73) Assignee: General Electric Company, Niskayuna, NY (US)

(21) Appl. No.: 10/065,892

(22) Filed: Nov. 27, 2002

(51) Int. Cl. A61B 5/055

(52) U.S. Cl. 600/410

(57) ABSTRACT

A method and system for detecting iron using magnetic resonance imaging (MRI) is provided. The method comprises acquiring magnetic resonance (MR) images by a selected pulse sequence to enhance brain iron deposits using a MRI system having a substantially high magnetic field strength and characterizing regions of interest within the MR images having statistically relevant quantities of iron deposits to indicate a given disease.

### BACKGROUND OF INVENTION

The invention relates to magnetic resonance imaging (MRI) and image processing methods. More particularly, the invention relates to detection of brain iron deposits using MRI and image processing techniques.

It has been known for some time that specific regions of the brain contain deposits of iron in a storage pool consisting of iron atoms in a mineral matrix associated with and largely surrounded by associated proteins. The total complex of mineralized iron and proteins is referred to as ferritin or in other cases as hemosiderin. It has also been recognized that these deposits are to some extent capable of being visualized on MR images because of the tendency of the magnetized iron atoms to alter the local magnetic field and to thereby to reduce the MR signal from protons in water molecules and other compounds in their vicinity of the iron deposits. This effect is referred to as iron-dependent shortening of the local T2 relaxation time. It is known that this effect is more prominent and more easily observed at higher magnetic field strengths. However, this imaging phenomenon has not been widely used for diagnostic purposes because of the difficulty in making diagnostic inferences due to the limited sensitivity of standard MR scanners and the complex and irregular shapes of the affected brain regions. Consequently, there is a need for an invention to improve the sensitivity of MR imaging to the presence of brain iron deposits and to improve the methods of analysis of the MR images to detect disease-related changes. One urgent need in neurology is an imaging method capable of detecting abnormal deposits in the brain, such as amyloid plaques and neurofibrillary tangles, which are associated with Alzheimer's disease and related diseases. It is known that iron in the form of ferritin or related proteinaceous compounds is often associated with these deposits. Although these deposits are often too small to be imaged as individual structures within the brain by MRI, the presence of several such deposits within an MR imaging voxel may lead to reduced overall signal strength for this voxel because of the iron content. Thus, by a process of signal averaging across a single voxel, this technique may be used to establish the presence of these pathological structures. Furthermore, a number of degenerative brain diseases (e.g., Parkinson's disease, Hallervorden Spatz disease and many others) have been found to be associated with increased regional iron deposition.

To date, most efforts to utilize brain-iron dependent contrast have utilized relatively thick slice (e.g., 3-5 mm), low-field (e.g., 1.5 T) images analyzed by visual inspection or by measurements of the image intensity variation or T2 relaxation of individual voxels. This method is cumbersome and time-consuming and, unless high-resolution imaging is used, local details of the iron distribution are not resolved.

Thus, there is a need for methods to perform MR imaging of brain iron deposition for use in the diagnosis of and monitoring the progression of neuro-degenerative brain diseases that overcome the deficiencies and problems described above. More particularly, there a need for improved sensitivity of MR imaging to detect the presence of brain iron deposits and to improve the methods of analyzing MR images to diagnose disease and detect disease related changes.

### SUMMARY OF INVENTION

In a first aspect, a method for detecting iron in the brain using magnetic resonance imaging (MRI) is provided. The method comprises acquiring magnetic resonance (MR) images by a selected pulse sequence to enhance brain iron deposits using a MRI system having a substantially high magnetic field strength and characterizing regions of interest within the MR images having statistically relevant quantities of brain iron deposits to indicate a given disease.

In a second aspect, a system for detecting iron in the brain using magnetic resonance imaging (MRI) is provided. The system comprises a magnetic resonance imaging device having a substantially high magnetic field strength and the device being adapted for acquiring a plurality of thin slice and T2-weighted magnetic resonance (MR) images and an image processor coupled to the imaging device and adapted for characterizing regions of interest within the MR images having iron deposits for use in at least one of diagnosis, prognosis, and prediction of progression of iron-dependent diseases.

### BRIEF DESCRIPTION OF DRAWINGS

The features and advantages of the present invention will become apparent from the following detailed description of the invention when read with the accompanying drawings in which:

FIG. 1 illustrates a simplified block diagram of a Magnetic Resonance Imaging system to which embodiments of the present invention are useful;

FIG. 2 is a schematic illustration of an exemplary embodiment of a method for segmenting MR images for use in analyzing iron deposits in accordance with methods of the present invention; and,

FIG. 3 is an exemplary illustration of MR images of brain iron taken at a magnetic field strength of 3 Tesla (3 T) to which embodiments of present invention are applicable.

### DETAILED DESCRIPTION

MRI scanners, which are used in various fields such as medical diagnostics, typically use a computer to create images based on the operation of a magnet, a gradient coil assembly, and a radio frequency coil(s). The magnet creates a uniform main magnetic field that makes nuclei, such as hydrogen atomic nuclei, responsive to radio frequency excitation. The gradient coil assembly imposes a series of pulsed, spatial magnetic fields upon the main magnetic field to give each point in the imaging volume a spatial identity corresponding to its unique set of magnetic fields during the imaging pulse sequence. The radio frequency coil(s) creates an excitation frequency pulse that temporarily creates an oscillating transverse magnetization that is detected by the radio frequency coil and used by the computer to create the image.

Generally, very high field strength is characterized as greater than 1.5 Tesla (1.5 T). In recent years, there has been an increase in usage of MRI systems at field strengths above the typical 1.5 Tesla. Research systems have been built as high as 8 Tesla. Systems are now commercially available at 3 Tesla and 4 Tesia. The systems are primarily used for research in functional MRI (fMRI) and human head related imaging and spectroscopy studies.

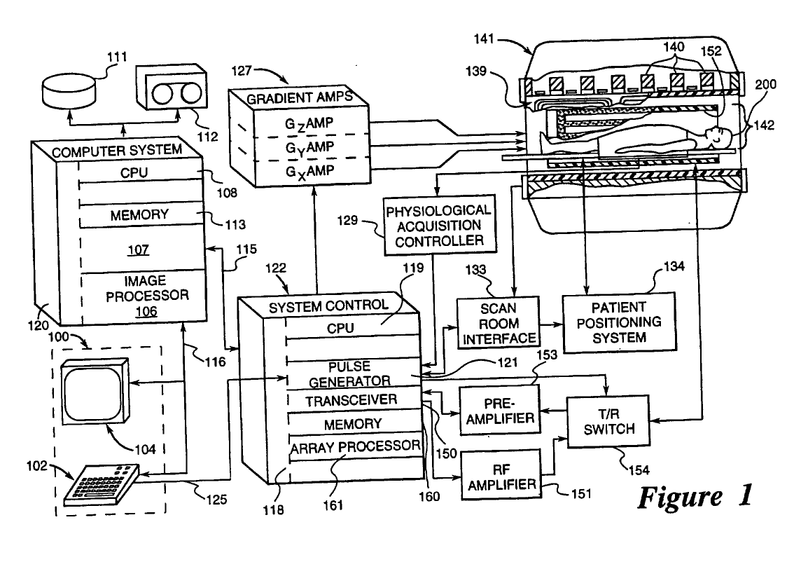

FIG. 1 illustrates a simplified block diagram of a system for producing images in accordance with embodiments of the present invention. In an embodiment, the system is a MR imaging system which incorporates the present invention. The MRI system could be, for example, a GE-Signa MR scanner available from GE Medical Systems, Inc., which is adapted to perform the method of the present invention, although other systems could be used as well.

The operation of the MR system is controlled from an operator console 100 which includes a keyboard and control panel 102 and a display 104. The console 100 communicates through a link 116 with a separate computer system 107 that enables an operator to control the production and display of images on the screen 104. The computer system 107 includes a number of modules that communicate with each other through a backplane. These include an image processor module 106, a CPU module 108, and a memory module 113, known in the art as a frame buffer for storing image data arrays. The computer system 107 is linked to a disk storage 111 and a tape drive 112 for storage of image data and programs, and it communicates with a separate system control 122 through a high speed serial link 115.

The system control 122 includes a set of modules connected together by a backplane. These include a CPU module 119 and a pulse generator module 121 which connects to the operator console 100 through a serial link 125. It is through this link 125 that the system control 122 receives commands from the operator which indicate the scan sequence that is to be performed. The pulse generator module 121 operates the system components to carry out the desired scan sequence. It produces data that indicate the timing, strength, and shape of the radio frequency (RF) pulses that are to be produced, and the timing of and length of the data acquisition window. The pulse generator module 121 connects to a set of gradient amplifiers 127, to indicate the timing and shape of the gradient pulses to be produced during the scan. The pulse generator module 121 also receives subject data from a physiological acquisition controller 129 that receives signals from a number of different sensors connected to the subject 200, such as ECG signals from electrodes or respiratory signals from a bellows. And finally, the pulse generator module 121 connects to a scan room interface circuit 133 (which receives signals from various sensors associated with the condition of the subject 200) and the magnet system. It is also through the scan room interface circuit 133 that a positioning device 134 receives commands to move the subject 200 to the desired position for the scan.

The gradient waveforms produced by the pulse generator module 121 are applied to a gradient amplifier system 127 comprised of Gₓ, G_{y} and G_{z} amplifiers. Each gradient amplifier excites a corresponding gradient coil in an assembly generally designated 139 to produce the magnetic field gradients used for position encoding acquired signals. The gradient coil assembly 139 forms part of a magnet assembly 141 which includes a polarizing magnet 140 and a whole-body RF coil 1152. Volume 142 is shown as the area within magnet assembly 141 for receiving subject 200 and includes a patient bore. As used herein, the usable volume of a MRI scanner is defined generally as the volume within volume 142 that is a contiguous area inside the patient bore where homogeneity of main, gradient and RF fields are within known, acceptable ranges for imaging. A transceiver module 150 in the system control 122 produces pulses that are amplified by an RF amplifier 151 and coupled to the RF coil 152 by a transmit/receive switch 154. The resulting signals radiated by the excited nuclei in the subject 200 may be sensed by the same RF coil 152 and coupled through the transmit/receive switch 154 to a preamplifier 153. The amplified MR signals are demodulated, filtered, and digitized in the receiver section of the transceiver 150. The transmit/receive switch 154 is controlled by a signal from the pulse generator module 121 to electrically connect the RF amplifier 151 to the coil 152 during the transmit mode and to connect the preamplifier 1153 during the receive mode. The transmit/receive switch 154 also enables a separate RF coil (for example, a head coil or surface coil) to be used in either transmit or receive mode. As used herein, “adapted to”, “configured” and the like refer to mechanical or structural connections between elements to allow the elements to cooperate to provide a described effect; these terms also refer to operation capabilities of electrical elements such as analog or digital computers or application specific devices (such as an application specific integrated circuit (ASIC)) that is programmed to perform a sequel to provide an output in response to given input signals.

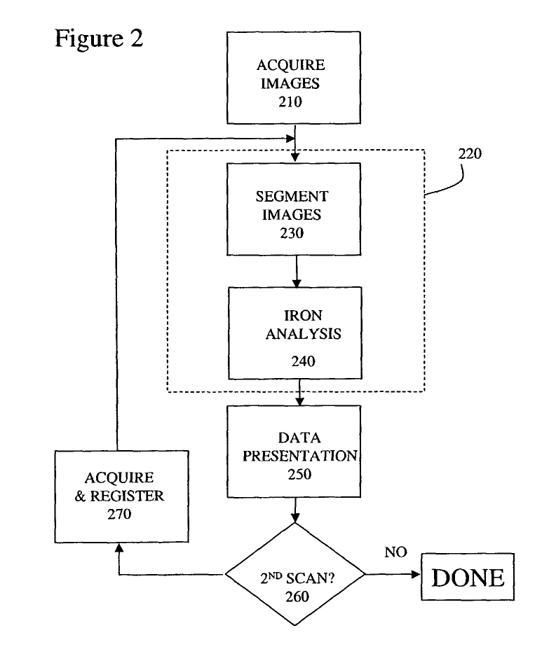

The MR signals picked up by the RF coil 152 are digitized by the transceiver module 150 and transferred to a memory module 160 in the system control 122. When the scan is completed and an entire array of data has been acquired in the memory module 160, an array processor 161 operates to Fourier transform the data into an array of image data. These image data are conveyed through the serial link 115 to the computer system 107 where they are stored in the disk memory 111. In response to commands received from the operator console 100, these image data may be archived on the tape drive 112, or they may be further processed by the image processor 106 and conveyed to the operator console 100 and presented on the display 104. Image processor 106 is further adapted to perform the image processing techniques which will be in greater detail below and with reference to FIG. 2. It is to be appreciated that a MRI scanner is designed to accomplish field homogeneity with given scanner requirements of openness, speed and cost.

As used herein, the term “very high field” refers to magnetic fields produced by the MRI system that are greater than about 1.5 Tesla. For embodiments of the invention the high field is desirably about 3 Tesla (3 T). Also, as used herein, “very high frequency” is considered to be the range of about 64 MHz to about 500 MHz, with a desired range between about 128 MHz and about 300 MHz. For embodiments of the invention, the high frequency is desirably at about 128 MHz.

All data gathered from multiple scans of the patient is to be considered one data set. Each data set can be broken up into smaller units, either pixels or voxels. When the data set is two-dimensional, the image is made up of units called pixels. A pixel is a point in two-dimensional space that can be referenced using two-dimensional coordinates, usually x and y. Each pixel in an image is surrounded by eight other pixels, the nine pixels forming a three-by-three square. These eight other pixels, which surround the center pixel, are considered the eight-connected neighbors of the center pixel. When the data set is three-dimensional, the image is displayed in units called voxels. A voxel is a point in three-dimensional space that can be referenced using three-dimensional coordinates, usually x, y and z. Each voxel is surrounded by twenty-six other voxels. These twenty-six voxels can be considered the twenty-six connected neighbors of the original voxel.

In embodiments of the present invention, high-resolution MR images are taken preferably at a magnetic field strength of 3 Tesla or more. These images may use a slice thickness of 1.5 mm or less. Any pulse sequence that produces a “T2-weighting” of the image intensity may be used. Generally speaking, the pulse sequence should balance achieving a high T2-weighting with the preservation of signal-to-noise-ratio. Pulse generator module 121 is adapted to produce T2-weighted images and to acquire substantially thin slice MR images for embodiments of the invention.

In an embodiment of the present invention, a method for detecting iron in the brain using magnetic resonance imaging (MRI) comprises the steps of acquiring magnetic resonance (MR) images by a selected pulse sequence to enhance brain iron deposits using a MRI system having a substantially high magnetic field strength and thereafter characterizing the regions of interest within the MR images having statistically relevant quantities of brain iron deposits to indicate a given disease. Generally, brain iron deposits are associated and indicative Alzheimer's disease, Parkinson's disease, Huntington's disease, Hallervorden Spatz disease, other neurodegenerative disorders, and other diseases of the central nervous system. Depending on the disease, there may be more or less statistically relevant brain iron to characterize the given disease. In an alternative embodiment, the characterizing of brain iron comprises measuring MR signal modifications produced by the brain deposits and using the signal modification in monitoring at least one of the progression of a given disease and response to therapeutic activity. Further, characterizing the brain iron comprises processing the regions of interest using computer-aided analysis based on image intensity, T2 values, intensity ratios and signal loss in order to enhance detection of brain iron within brain substructures. Additionally, characterizing further comprises producing volumetric measurements of the regions of interest, wherein the volumetric measurements are used in quantifying progression of the given disease and/or monitoring response to therapy.

In a further embodiment, the steps of acquiring and characterizing are repeated in at least one successive or serial examination, typically at a later time, of a given subject for measuring progression of the disease and measuring response to therapy. Additionally, the method includes interfacing with a data source, such as same subject examination data, clinical population data for the given disease and bioinformatic data, in order for the image processor to perform comparisons of the regions of interest with data from the respective data sources. As more and more is known about neurodegenerative disease and corresponding relevant iron information, then comparison with the data sources would enable disease staging, predictive modeling and other such tracking of the disease for a given patient.

Referring to FIG. 2, an embodiment for segmenting MR images is provided that segments and quantifies brain structures, and most specifically brain iron deposits, from T2 dual echo MR images. As used herein, “T2”, “T2 parameter” and the like refer to the time constant, or alternatively spin-spin relaxation time, T2 that is well known in the art of MR imaging. T2 is the time measurement for a given nuclei to return to be uniformly distributed around the static magnetic field (referred to as “B”) once the RF pulse sequence is completed in the MR scan. There is a T2 value associated with a given tissue type or brain structure, thus the T2 value is useful in distinguishing selected tissue types in a MR image. It is known that T2 relaxation time is shortened in the presence of iron deposits. This effect is referred to as iron-dependent shortening of the local T2 relaxation time. Further, the given T2 value may be visualized differently between dual echo images. For example, the cerebrospinal fluid (CSF) typically has higher values in the second echo and extra cranial tissues such as the face have higher values in the first echo.

The input to the method shown in FIG. 2 are images acquired at step 210 by MRI scanning, for example on a MR scanner having a 3 T magnetic field strength, for example a commercially available 3 T MRI system from General Electric. The dual echo was acquired by known methods using T2 spin echo pulse sequence. In an exemplary embodiment, the first echo, is a proton density weighted (PDW) pulse, and the second echo is a T2 weighted (T2W) pulse. It is to be appreciated by those skilled in the art that other modified pulse sequences may also applicable to methods described herein.

Referring further to FIG. 2, desirably, the acquired images should cover a contiguous region of the subject's brain inclusive of regions of interest that contain the iron deposits of interest. Under most clinical conditions these regions would include the basal ganglia, the thalamus, the mid-brain, the medial temporal lobe and specific regions of the cerebral cortex and the cerebellum. The images are submitted to computer-aided analysis 220 to characterize the regions of iron-deposition. Computer-aided analysis may include various known segmentation and computer analysis algorithms, shown as 230 and 240. This characterization may be made on the basis of a number of image-related parameters. Segmentation 230, part of the analysis, can be any of the many known segmentation techniques, such as T2 weighting, region growing, or intensity thresholds. The iron analysis step 240 can be performed a number of ways. In an embodiment, the presence of iron deposits is detected by loss of signal intensity on T2-weighted images. The computer analysis of these regions can be performed by classifying regions in terms of image intensity (which is reduced for iron-rich regions on late echo images), calculated T2-values (which are reduced in iron-rich regions), ratio images where the image intensity in late-echo images is divided by the intensity in early-echo images or by other mathematical procedures which display the loss of signal intensity produced by iron deposits. In a further embodiment for iron analysis, the computer-processed images acquired by segmentation can be subjected to further computer analysis to determine parameters such as the volumetric measurements of the individual iron-containing brain regions, the local variability in iron deposition (such as the standard deviation of the intensity of neighboring voxels) and the total enhancement of signal loss (compared to iron-free regions) which is related to the regional concentration and state of aggregation of the iron particles within the brain. The result of the computer analysis of these high-resolution, iron-weighted images is a quantitative report or other data presentation 250 on the volume of the iron-rich regions (e.g., the substantia nigra and the globus pallidus), the extent of iron deposition (as measured by various quantitative determinations of the regional signal loss—such as local T2). It is to be appreciated that there are various embodiments for data presentation 250, for example images with color-coded areas showing iron deposits or alternatively volumetric measurements indicating the extent of iron deposits.

A number of degenerative brain diseases (e.g., Parkinson's disease, Hallervorden Spatz disease and many others) have been found to be associated with increased regional iron deposition. With the high resolution MR imaging and computer analysis, as described herein, it is likely that many new brain regions with high iron depositions will be identified and characterized, thereby extending this diagnostic technique to additional disease states. Furthermore, the use of computer-generated information, such as volumetric analysis of affected brain regions and the ability to track this parameter in serial studies of a given patient by use of computer image registration techniques, provides a means of quantifying the progression of disease and the response to therapy.

Referring further to FIG. 2, serial studies of a given patient would require a second or successive scan 260 by the MRI system at a later time. When a successive scan is performed, then the acquisition of the successive image also requires some registration (Acquire and Register step 270) to register the successive scan image data with the previous image data. Additionally, the registration may require registration to a given MR scanner in order to calibrate scanner-related variations of the successive scan. It is to appreciated that there are many known registration techniques available to one skilled in the art of MR imaging that may be used to register the images of successive scans to compensate for time and scanner-related variations.

Once image data is acquired and analyzed by the process described above, the image data may be used for various aspects of disease diagnosis and tracking. For example, quantitative characterization of iron deposits will enable a physician to track the disease progression or response to therapy of a patient. The acquisition and characterization are repeated and patient image data can be followed serially in a given patient through the use of image registration techniques. Another advantage is the possibility of quantifying the spatial extent and intensity of iron-deposition in and thereby providing quantitative volumetric measures of irregularly shaped brain nuclei. The method provides a convenient, computer-assisted tracking of changes in iron deposition associated with disease onset, progression and therapy.

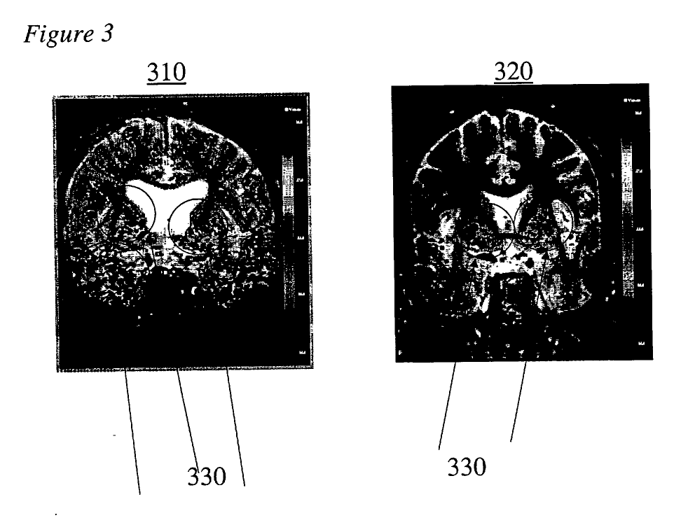

FIG. 3 shows an exemplary illustration of MR images of brain iron taken at a magnetic field strength of 3 Tesla (3 T) to which embodiments of present invention are applicable. Image 310 is a MR image of a brain of a subject with Alzheimer's disease having a number of speckled regions 330 which are regions having shortened T2 indicating the presence of iron. Image 320 is a MR image of a normal brain, in which also has some speckled regions 330 but substantially less in number and distribution than the AD subject. Thus, through the use of methods in accordance with the present invention described above, it is possible to detect brain iron within brain structures which provides the ability to diagnose and detect disease related changes.

Embodiments described above focused on methods to enhance the detection of brain iron for the purpose of diagnosing and detecting neurodegenerative diseases. However, it is to be appreciated that the methods of the present invention would be similarly applicable to imaging structures outside the brain, for example the liver. One skilled in the art would find the methods of acquiring and characterizing to enhance iron deposits could be applied similarly to diseases such as hereditary hemochromatosis and secondary hemochromatosis which lead to an iron overload in the liver and other tissues. Similarly, the methods of the present invention may be applied to diseases that are indicated by shortened T2. For example, there is evidence that shortened T2 is present in images of patients having atherosclerotic plaque, such as in atherosclerotic brain disease or atherosclerotic cardiovascular disease. It is to be appreciated that applying methods of the present invention would provide predictive value for the potential of developing a stroke, heart disease or further disease progression.

While the preferred embodiments of the present invention have been shown and described herein, it will be obvious that such embodiments are provided by way of example only. Numerous variations, changes and substitutions will occur to those of skill in the art without departing from the invention herein. Accordingly, it is intended that the invention be limited only by the spirit and scope of the appended claims.

1. A method for detecting iron in the brain using magnetic resonance imaging (MRI) comprising:
acquiring magnetic resonance (MR) images by a selected pulse sequence to enhance brain iron deposits using a MRI system having a substantially high magnetic field strength; and,
characterizing regions of interest within the MR images having statistically relevant quantities of brain iron deposits to indicate a given disease.

2. The method of claim 1 wherein the selected pulse sequence is adapted to acquire a plurality of T2-weighted and substantially thin slice MR images.

3. The method of claim 1 wherein the characterizing step comprises measuring MR signal modifications produced by the brain deposits and using the signal modification in monitoring at least one of the progression of a given disease and response to therapeutic activity.

4. The method of claim 1 the characterizing step comprises processing the regions of interest using computer-aided analysis based on at least one of image intensity, T2 values, intensity ratios and signal loss in order to enhance brain iron within brain substructures.

5. The method of claim 4 further comprising producing volumetric measurements of the regions of interest, wherein the volumetric measurements are used in at least one of quantifying progression of the given disease and monitoring response to therapy.

6. The method of claim 1 wherein the brain iron deposits are indicative of diseases comprising Alzheimer's disease, Parkinson's disease, Huntington's disease, Hallervorden Spatz disease, other neurodegenerative diseases and atherosclerotic diseases.

7. The method of claim 1 wherein the substantially high magnetic field strength is about 1.5 Tesla (1.5 T) or greater.

8. The method of claim 2 wherein the thin slice is about 1.5 mm or less.

9. The method of claim 1 further comprising repeating the acquiring and characterizing steps in at least one successive examination of a given subject for at least one of measuring progression of the disease and measuring response to therapy.

10. The method of claim 1 wherein the characterizing step further comprises interfacing with a data source, the data source comprising at least one of same subject examination data, clinical population data for the given disease and bioinformatic data, to perform comparisons of the regions of interest with data from the respective data source.

11. A method for detecting iron using magnetic resonance imaging (MRI) comprising:
acquiring a plurality of thin slice and T2-weighted magnetic resonance (MR) images with a substantially high magnetic field strength;
characterizing regions of interest within the MR images having iron deposits for use in at least one of diagnosis, prognosis, and prediction of progression of iron-dependent diseases.

12. The method of claim 111 further comprising analyzing the characterized regions of interest having iron deposits using computer analysis.

13. The method of claim 11 wherein acquiring step comprises at least one pulse sequence adapted to acquire substantially thin slice images.

14. The method of claim 11 wherein acquiring step comprises at least one pulse sequence adapted to produce T2-weighting of image intensity.

15. The method of claim 11 wherein the characterizing step comprises:
processing the MR images using computer-aided analysis to characterize regions of iron-deposition; and,
producing volumetric measurements of the regions of iron-deposition.

16. The method of claim 15 wherein the volumetric measurements are used to quantify progression of the disease.

17. The method of claim 15 wherein the volumetric measurements are used to measure response to therapy.

18. The method of claim 11 further comprising repeating the acquiring and characterizing steps in at least one successive examination of a given subject for at least one of measuring progression of the disease and measuring response to therapy.

19. The method of claim 11 wherein the iron-dependent diseases comprise Alzheimer's disease, Parkinson's disease, Huntington's disease, Hallervorden Spatz disease, other neurodegenerative diseases, liver diseases and atherosclerotic diseases.

20. The method of claim 11 wherein the characterizing step comprises measuring signal alterations produced by iron deposits.

21. The method of claim 11 wherein the substantially high magnetic field strength is about 1.5 Tesla (1.5 T) and greater.

22. The method of claim 11 wherein a thin slice is about 1.5 mm or less.

23. The method of claim 11 wherein the characterizing step further comprises interfacing with a data source, the data source comprising at least one of same subject examination data, clinical population data for the given disease and bioinformatic data, to perform comparisons of the regions of interest with data from the respective data source.

24. The method of claim 11 wherein the characterizing step comprises:
segmenting the MR images into a plurality of selected substructures and iron based on respective T2 relaxation times corresponding to each of the substructures and iron; and,
analyzing the iron for at least one of volume, intensity and signal loss.

25. The method of claim 24 wherein the MR images are acquired by employing a dual echo pulse sequence comprising proton density weighted (PDW) and T2 weighted images.

26. The method of claim 24 wherein the analyzing comprises computer-aided analysis of the iron.

27. The method of claim 24 wherein the analyzing comprises regional analysis of the iron and the regional analysis comprises at least one of histograms, intensity and statistical analysis.

28. A system for detecting iron using magnetic resonance imaging (MRI) comprising:
a magnetic resonance imaging device having a substantially high magnetic field strength and the device being adapted for acquiring a plurality of thin slice and T2-weighted magnetic resonance (MR) images;
an image processor coupled to the imaging device and adapted for characterizing regions of interest within the MR images having iron deposits for use in at least one of diagnosis, prognosis, and prediction of progression of iron-dependent diseases.

29. The system of claim 28 wherein the substantially high magnetic field strength is about 1.5 Tesla (1.5 T) and greater.

30. The system of claim 28 wherein the thin slice is about 1.5 mm or less.

31. The system of claim 28 wherein the iron-dependent diseases comprise Alzheimer's Disease, Parkinson's Disease, Huntington's Disease, Hallervorden Spatz disease, other neurodegenerative diseases, liver diseases and atherosclerotic diseases.

32. The system of claim 28 further comprising an interface unit coupled to the image processor for interfacing with a data source to perform comparisons of the regions of interest with data from the respective data source, the data source comprising at least one of same subject examination data, clinical population data for the given disease and bioinformatic data.

33. The system of claim 28 wherein the image processor is adapted to perform at least one of volumetric measurements, regional analysis, computer-aided analysis and segmentation of the regions of interest.
